(19) United States
(12) Patent Application Publication
(10) Pub. No.: US 2019/0177295 A1
(43) Pub. Date: Jun. 13, 2019

(54) PROCESS FOR THE PREPARATION OF A 1,3-DISUBSTITUTED PYRAZOLE COMPOUND

(71) Applicant: VERTEX PHARMACEUTICALS INCORPORATED, Boston, MA (US)

(72) Inventors: Cavan McKeon Bligh, Melrose, MA (US); John J. Court, Littleton, MA (US); Jianglin Liang, Needham, MA (US); Hardwin O'Dowd, Cambridge, MA (US); Stefanie Roeper, Medford, MA (US); Michael P. Ryan, Charlestown, MA (US)

(21) Appl. No.: 16/213,282

(22) Filed: Dec. 7, 2018

Related U.S. Application Data
(60) Provisional application No. 62/596,386, filed on Dec. 8, 2017.
(60) Provisional application No. 62/682,633, filed on Jun. 8, 2018.

(51) Int. Cl. C07D 401/04; C07C 53/44

(52) U.S. Cl. CPC C07D 401/04; B01D 9/005; C07C 53/44

(57) ABSTRACT

The disclosure provides a process for preparing a compound of formula (I)

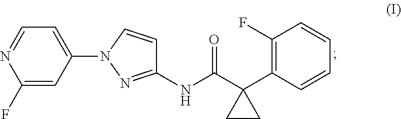 [SMILES: O=C(Nc1ccn(-c2ccnc(F)c2)n1)C1(c2ccccc2F)CC1]

or a pharmaceutically acceptable salt thereof.

### BACKGROUND

Adrenoleukodystrophy (ALD) (also known as X-linked adrenoleukodystrophy or X-adrenoleukodystrophy (X-ALD)) patients suffer from debilitating, and often fatal, neurological effects and adrenal insufficiency often associated with one or more mutations in the ATP binding cassette transporter D1 (ABCD1) gene. ABCD1 plays a critical role in very long chain fatty acid (VLCFA) degradation and, as such, ALD patients typically have elevated VLCFA levels that are thought to be causative of the pathology in ALD. The prevalence of ALD is 1 in 20,000 to 50,000 individuals worldwide. The overall incidence of ALD is estimated to be 1 in 17,000 newborns (males and females). In males there are two predominant phenotypes: cerebral ALD (CALD) and adrenomyeloneuropathy (AMN). CALD is the more extreme form, which presents with rapidly progressive inflammatory demyelination of the brain, leading to rapid cognitive and neurological decline. If untreated, CALD patients die within approximately 2 years of symptom onset. Over the course of their lifetime, approximately 60% of males with ALD will develop CALD, most frequently between the ages of about 3 and about 12 (35 to 40%), with continued (albeit decreasing) risk during adulthood. Adult males with ALD will develop adrenomyeloneuropathy (AMN), a slowly progressive axonopathy with first symptoms appearing around 20 to 30 years of age. AMN is characterized by chronic myelopathy with progressive spastic paraparesis, sensory ataxia, sphincter dysfunction and impotence, commonly associated with primary adrenocortical and/or testicular insufficiency. Approximately 7,000 to 10,000 males in the US and EU combined will develop AMN. Women with ALD are also affected and not merely carriers: >80% of these individuals develop signs and symptoms of myelopathy by the age of 60 years. Approximately 12,000 to 15,000 women in the US and EU combined will eventually develop AMN. Female ABCD1 heterozygotes exhibit approximately half the plasma VLCFA elevation observed in males, never develop the cerebral form of the disease, and develop more modest, but debilitating, AMN-like symptoms later in life. Therefore, about a 50% to about a 75% reduction in VLCFA levels relative to a patient's baseline VLCFA level may be sufficient to prevent cerebral ALD, delay onset, and/or reduce disease severity and progression.

Mutations in any of three separate genes in the VLCFA degradation pathway have been associated with VLCFA accumulation and demyelinating diseases in humans. In addition to mutations in ABCD1, mutations in Acyl-CoA oxidase (ACOX1) or D-Bifunctional protein (DBP) also are associated with accumulation of VLCFA and demyelinating disorders, supporting the hypothesis that increased VLCFA cause the underlying pathophysiology of ALD.

1-(2-fluorophenyl)-N-(1-(2-fluoropyridin-4-yl)-1H-pyrazol-3-yl)cyclopropane-1-carboxamide has been found to reduce VLCFA levels in cell-based assays and in model organisms, suggesting that the compound is a therapeutic candidate for ALD.

### SUMMARY

In one aspect, the disclosure relates to a process for preparing a compound of formula (I)

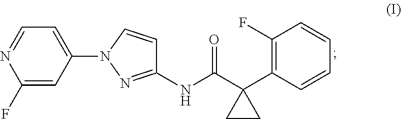 [SMILES: O=C(Nc1ccn(-c2ccnc(F)c2)n1)C1(c2ccccc2F)CC1]

or a pharmaceutically acceptable salt thereof, comprising:

reacting a compound of formula (II)

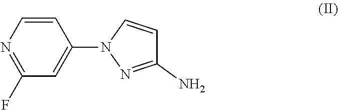 [SMILES: Nc1ccn(-c2ccnc(F)c2)n1]

with a compound of formula (III)

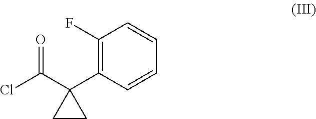 [SMILES: O=C(Cl)C1(c2ccccc2F)CC1]

in the presence of a suitable base to afford the compound of formula (I); and

optionally reacting the compound of formula (I) with a suitable acid to afford a pharmaceutically acceptable salt of the compound of formula (I).

In another aspect, the disclosure relates to a process for preparing a compound of formula (I)

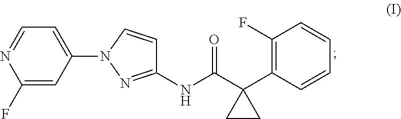 [SMILES: O=C(Nc1ccn(-c2ccnc(F)c2)n1)C1(c2ccccc2F)CC1]

or a pharmaceutically acceptable salt thereof, comprising:

reacting a compound of formula (V)

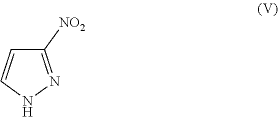

with a compound of formula (VI)

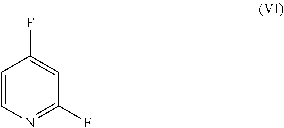

in the presence of a suitable base to afford a compound of formula (IV)

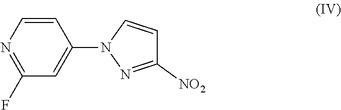 [SMILES: O=[N+]([O-])c1ccn(-c2ccnc(F)c2)n1]

and transforming the compound of formula (IV) to the compound of formula (I), or a pharmaceutically acceptable salt thereof.

In another aspect, the disclosure relates to a compound of formula (II), (III), or (IV).

### DETAILED DESCRIPTION

The disclosure relates to processes for preparing a compound (or a pharmaceutically acceptable salt thereof) useful for reducing VLCFA levels in a patient. The compound may be represented by formula (I):

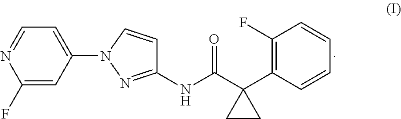 [SMILES: O=C(Nc1ccn(-c2ccnc(F)c2)n1)C1(c2ccccc2F)CC1]

A process for preparing the compound of formula (I) is set forth in Scheme 1. The process outlined in Scheme 1 may be conducted as a batch process, with isolation of the product after each step, or as a continuous flow process. When the process is conducted as a continuous flow process, each step may be performed in the same solvent (e.g., THF) in order to avoid the need for solvent removal or exchange between steps.

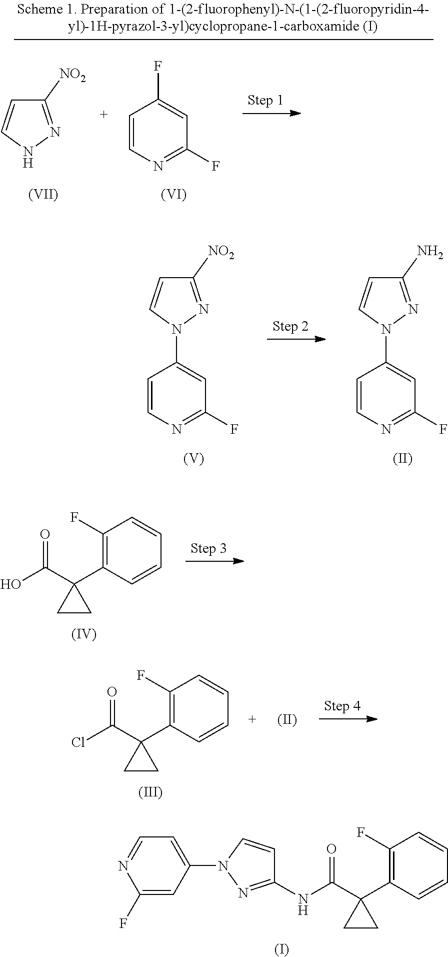

In Step 1, 3-nitro-1H-pyrazole (VII) is treated with 2,4-difluoropyridine (VI) in the presence of a base. A nucleophilic aromatic substitution reaction affords 2-fluoro-4-(3-nitro-1H-pyrazol-1-yl)pyridine (V).

The base used in the nucleophilic aromatic substitution reaction (Step 1) may be any suitable base known to those skilled in the art. Examples of suitable bases include inorganic hydrides (e.g., NaH), inorganic phosphates and carbonates (e.g., K₃PO₄, K₂CO₃, and Cs₂CO₃), secondary or tertiary amines (e.g., Hünig's base, tetramethylpiperidine), and guanidines (e.g., tetramethylguanidine, and 7-Methyl-1,5,7-triazabicyclo[4.4.0]dec-5-ene). It was discovered that the inorganic phosphate and carbonate bases (particularly K₃PO₄) provided a favorable combination of scalability and fast reaction rate, while minimizing the formation of a side product resulting from bis-addition of 3-nitro-1H-pyrazole (VII) to 2,4-difluoropyridine (VI) (“bis-adduct side product”).

The nucleophilic aromatic substitution reaction (Step 1) may be conducted in any suitable solvent known to those skilled in the art. Examples of suitable solvents include polar aprotic solvents (e.g., N,N-dimethylformamide (DMF), N-methylpyrrolidone (NMP), dimethylsulfoxide (DMSO), acetonitrile, and 1,3-dimethyl-2-imidazolidinone (DMI)), dichloromethane, neat tetramethylguanidine (TMG), and 1-butyl-3-methylimidazolium tetrafluoroborate.

The nucleophilic aromatic substitution reaction (Step 1) may be conducted at any suitable temperature. For example, the temperature may be between 0° C. and 35° C., or between 0° C. and room temperature, or between 0° C. and 15° C. It was discovered that formation of the bis-adduct side product is minimized when the reaction is conducted at a temperature between 0° C. and 15° C.

The product of the nucleophilic aromatic substitution reaction (Step 1), 2-fluoro-4-(3-nitro-1H-pyrazol-1-yl)pyridine (V), may be purified by any suitable method known to those skilled in the art. For example, 2-fluoro-4-(3-nitro-1H-pyrazol-1-yl)pyridine (V) may be separated from the bis-adduct side product by recrystallization from a mixture of dichloromethane and methanol. The ratio of dichloromethane to methanol may be between 1:3 and 1:2 (v/v). It was discovered that dissolution of the crude product in a mixture of dicloromethane and methanol, followed by cooling, results in precipitation of the bis-adduct side product.

In Step 2, 2-fluoro-4-(3-nitro-1H-pyrazol-1-yl)pyridine (V) is treated with H₂ in the presence of a catalyst. Reduction of the nitro substituent on 2-fluoro-4-(3-nitro-1H-pyrazol-1-yl)pyridine (V) affords 1-(2-fluoropyridin-4-yl)-1H-pyrazol-3-amine (II).

The catalyst used in the reduction reaction (Step 2) may be any suitable hydrogenation catalyst known to those skilled in the art. Examples of suitable hydrogenation catalysts include palladium catalysts (e.g., palladium hydroxide on carbon (Pd(OH)₂/C, Pearlman's Catalyst), and palladium on carbon (Pd/C)) and platinum catalysts (e.g., platinum on carbon (Pt/C)). A variety of catalyst loadings (e.g., 2 mol %, 0.3 mol %, 0.1 mol %, and 0.05 mol %) are suitable, depending on the activity of the catalyst. It was discovered that platinum catalysts, and particularly Pt/C, provided favorable impurity profiles and reaction rates at low catalyst loadings. In a screening reaction, it was found that a reaction conducted at 50° C. with 2 mol % of Pt/C was complete within 2 hours and provided 95.4% selectivity for 1-(2-fluoropyridin-4-yl)-1H-pyrazol-3-amine (II). A reaction conducted at 50° C. with 0.05 mol % of Pt/C was complete in 1.5 hours and provided 92.93% selectivity for 1-(2-fluoropyridin-4-yl)-1H-pyrazol-3-amine (II).

The hydrogen used in the reduction reaction (Step 2) may be introduced as molecular hydrogen (H₂) or may be generated in situ. Examples of suitable reagents known to form hydrogen in situ include ammonium formate.

The reduction reaction (Step 2) may be conducted in any suitable solvent known to those skilled in the art. Examples of suitable solvents include tetrahydrofuran, dioxane, methanol, and mixtures thereof.

The reduction reaction (Step 2) may be conducted at any suitable temperature. For example, the temperature may be between 0° C. and 100° C., about room temperature, about 50° C., or about 70° C.

The product of the reduction reaction (Step 2), 1-(2-fluoropyridin-4-yl)-1H-pyrazol-3-amine (II), may be purified by any suitable method known to those skilled in the art. For example, 1-(2-fluoropyridin-4-yl)-1H-pyrazol-3-amine (II) may be purified by recrystallization from a mixture of isopropyl alcohol and water. It was discovered that dissolution of the crude product in a mixture of isopropyl alcohol and water at a ratio between 3:1 and 1.5:1 (v/v), followed by cooling and introduction of additional water, results in precipitation of 1-(2-fluoropyridin-4-yl)-1H-pyrazol-3-amine (II). Alternatively, 1-(2-fluoropyridin-4-yl)-1H-pyrazol-3-amine (II) may be purified by trituration in methanol.

In Step 3, 1-(2-fluorophenyl)cyclopropane-1-carboxylic acid (IV) is treated with thionyl chloride or oxalyl chloride to afford 1-(2-fluorophenyl)cyclopropanecarbonyl chloride (III). The reaction may be conducted neat, or in any suitable solvent. Suitable solvents include toluene, dichloromethane, dioxane, and tetrahydrofuran.

In Step 4, 1-(2-fluorophenyl)cyclopropanecarbonyl chloride (III) is treated with 1-(2-fluoropyridin-4-yl)-1H-pyrazol-3-amine (II) in the presence of a base. Amide formation affords 1-(2-fluorophenyl)-N-(1-(2-fluoropyridin-4-yl)-1H-pyrazol-3-yl)cyclopropane-1-carboxamide (I).

The base used in the amide formation reaction (Step 4) may be any suitable base known to those skilled in the art. Examples of suitable bases include trialkylamines (e.g, triethylamine, N-methylmorpholine, and Hünig's base) and aromatic amines (e.g., pyridine and 2,6-lutidine).

The amide formation reaction (Step 4) may be conducted in any suitable solvent known to those skilled in the art. Examples of suitable solvents include ethereal solvents (e.g., tetrahydrofuran and 2-m ethyltetrahydrofuran).

The amide formation reaction (Step 4) may be conducted at any suitable temperature. For example, the amide formation reaction may be conducted at a temperature between 0° C. and 50° C., or between 0° C. and 35° C., or between 5° C. and room temperature, or between 10° C. and 20° C.

The product of the amide formation reaction (Step 4), 1-(2-fluorophenyl)-N-(1-(2-fluoropyridin-4-yl)-1H-pyrazol-3-yl)cyclopropane-1-carboxamide (I), may be purified by any suitable method known to those skilled in the art. For example, 1-(2-fluorophenyl)-N-(1-(2-fluoropyridin-4-yl)-1H-pyrazol-3-yl)cyclopropane-1-carboxamide (I) may be separated from unreacted 1-(2-fluoropyridin-4-yl)-1H-pyrazol-3-amine (II) by recrystallization from a mixture of isopropyl alcohol and water or from a mixture of isopropyl acetate and heptane. It was discovered that dissolution of the crude product in warm isopropyl alcohol, followed by addition of water and cooling, results in precipitation of 1-(2-fluorophenyl)-N-(1-(2-fluoropyridin-4-yl)-1H-pyrazol-3-yl)cyclopropane-1-carboxamide (I). Alternatively, 1-(2-fluorophenyl)-N-(1-(2-fluoropyridin-4-yl)-1H-pyrazol-3-yl)cyclopropane-1-carboxamide (I) may be purified by column chromatography on a silica gel column.

Subsequent to Step 4, 1-(2-fluorophenyl)-N-(1-(2-fluoropyridin-4-yl)-1H-pyrazol-3-yl)cyclopropane-1-carboxamide (I) may be treated with a suitable acid to afford a pharmaceutically acceptable salt of 1-(2-fluorophenyl)-N-(1-(2-fluoropyridin-4-yl)-1H-pyrazol-3-yl)cyclopropane-1-carboxamide (I).

As an alternative to Steps 3 and 4, 1-(2-fluoropyridin-4-yl)-1H-pyrazol-3-amine (II) and 1-(2-fluorophenyl)cyclopropane-1-carboxylic acid (IV) may be coupled directly using an amide coupling reagent (e.g., propylphosphonic anhydride (T3P), carbonyl diimidazole (CDI), 1-[Bis(dimethylamino)methylene]-1H-1,2,3-triazolo[4,5-b]pyridinium 3-oxid hexafluorophosphate (HATU), or 1-Ethyl-3-(3-dimethylaminopropyl)carbodiimide (EDCI) under conditions familiar to those skilled in the art.

### EMBODIMENTS

In one aspect, the disclosure relates to a process for preparing a compound of formula (I)

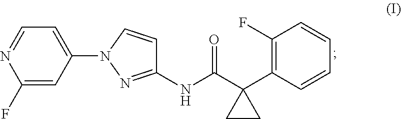 [SMILES: O=C(Nc1ccn(-c2ccnc(F)c2)n1)C1(c2ccccc2F)CC1]

comprising:

coupling a compound of formula (II)

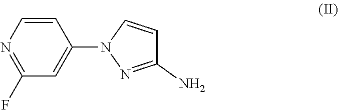 [SMILES: Nc1ccn(-c2ccnc(F)c2)n1]

with a compound of formula (III)

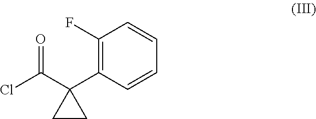 [SMILES: O=C(Cl)C1(c2ccccc2F)CC1]

to afford the compound of formula (I).

In one aspect, the disclosure relates to a process for preparing a compound of formula (I)

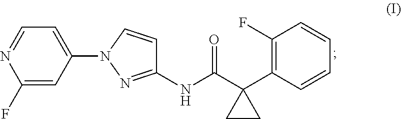 [SMILES: O=C(Nc1ccn(-c2ccnc(F)c2)n1)C1(c2ccccc2F)CC1]

or a pharmaceutically acceptable salt thereof, comprising:

reacting a compound of formula (II)

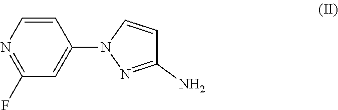 [SMILES: Nc1ccn(-c2ccnc(F)c2)n1]

with a compound of formula (III)

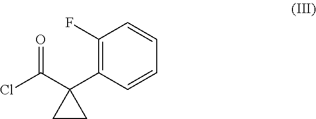 [SMILES: O=C(Cl)C1(c2ccccc2F)CC1]

in the presence of a suitable base to afford the compound of formula (I); and

optionally reacting the compound of formula (I) with a suitable acid to afford a pharmaceutically acceptable salt of the compound of formula (I).

In some embodiments, the process is for preparing the compound of formula (I) in non-salt form and does not include the optional step of reacting the compound of formula (I) with a suitable acid to afford a pharmaceutically acceptable salt of the compound of formula (I).

In some embodiments, the base is a trialkylamine. In some embodiments the trialkylamine is triethylamine.

In some embodiments, said reacting a compound of formula (II) with a compound of formula (III) is performed in tetrahydrofuran.

In some embodiments, the compound of formula (I) is purified by recrystallization. In some embodiments, the compound of formula (I) is purified by recrystallization from a mixture of isopropyl alcohol and water. In some embodiments, the compound of formula (I) is purified by recrystallization from a mixture of isopropyl acetate and heptane. In some embodiments, the recrystallization comprises dissolving the compound of formula (I) in isopropyl alcohol, adding water to the resulting solution, and then separating the compound of formula (I) as a solid. In come embodiments, isopropyl alcohol and water are present in a ratio of between about 2:1 and about 1:2 by volume when the compound of formula (I) is separated. In some embodiments, the compound of formula (I) purified by recrystallization contains no more than 5 wt. %, 4 wt. %, 3 wt. %, 2 wt. %, 1 wt. %, 5000 ppm, 4000 ppm, 3000 ppm, 2000 ppm, 1000 ppm, 500 ppm, 250 ppm, 100 ppm, 50 ppm, 25 ppm, or 20 ppm of the compound of formula (II).

In some embodiments, the compound of formula (II) is obtained by reacting a compound of formula (IV)

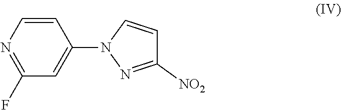 [SMILES: O=[N+]([O-])c1ccn(-c2ccnc(F)c2)n1]

with H₂ in the presence of a suitable hydrogenation catalyst to afford the compound of formula (II).

In some embodiments, the hydrogenation catalyst is a heterogeneous platinum catalyst. In some embodiments, the platinum catalyst is platinum on carbon.

In some embodiments, said reacting a compound of formula (IV) with H₂ is performed in a mixture of tetrahydrofuran and methanol.

In some embodiments, said H₂ is introduced in the form of H₂ gas.

In some embodiments, the compound of formula (II) is purified by recrystallization. In some embodiment, the compound of formula (II) is purified by recrystallization from a mixture of isopropyl alcohol and water. In some embodiments, the recrystallization comprises dissolving the compound of formula (II) in isopropyl alcohol, adding water to the resulting solution, and then separating the compound of formula (II) as a solid. In some embodiments, isopropyl alcohol and water are present in a ratio of between about 1:1 and about 3:1 by volume when the compound of formula (II) is separated.

In some embodiments, the compound of formula (IV) is obtained by reacting a compound of formula (V)

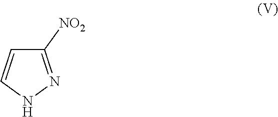

with a compound of formula (VI)

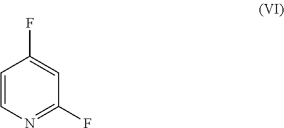

in the presence of a suitable base to afford the compound of formula (IV).

In some embodiments, the base is an inorganic phosphate or carbonate. In some embodiments, the base is K₃PO₄. In some embodiments, said reacting a compound of formula (V) with a compound of formula (VI) is performed in a polar aprotic solvent. In some embodiments, the polar aprotic solvent comprising DMF.

In some embodiments, said reacting a compound of formula (V) with a compound of formula (VI) is performed at a temperature between 0° C. and 50° C., 0° C. and 30° C., 0° C. and 15° C., or 0° C. and 12° C.

In some embodiments, 2,4-bis(3-nitro-1H-pyrazol-1-yl)pyridine is separated from the compound of formula (IV) by recrystallization. In some embodiments, the recrystallization is from a mixture of dichloromethane and methanol. In some embodiments, the recrystallization comprises dissolving the compound of formula (IV) in a mixture of dichloromethane and methanol, cooling the resulting solution, and then separating 2,4-bis(3-nitro-1H-pyrazol-1-yl)pyridine as a solid. In some embodiments, the dichloromethane and methanol are present in a ratio of between 1:2 and 1:3 by volume when the 2,4-bis(3-nitro-1H-pyrazol-1-yl)pyridine is separated.

In another aspect, the disclosure relates to a process for preparing a compound of formula (I)

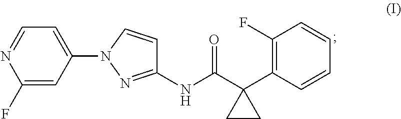 [SMILES: O=C(Nc1ccn(-c2ccnc(F)c2)n1)C1(c2ccccc2F)CC1]

or a pharmaceutically acceptable salt thereof, comprising:

reacting a compound of formula (V)

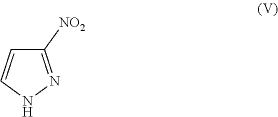

with a compound of formula (VI)

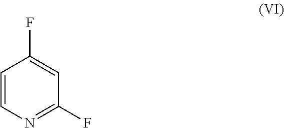

in the presence of a suitable base to afford a compound of formula (IV)

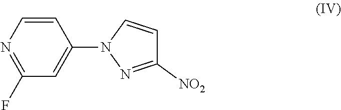 [SMILES: O=[N+]([O-])c1ccn(-c2ccnc(F)c2)n1]

and transforming the compound of formula (IV) to the compound of formula (I), or a pharmaceutically acceptable salt thereof.

In some embodiments, the base is an inorganic phosphate or carbonate. In some embodiments, the base is K₃PO₄.

In some embodiments, said reacting a compound of formula (V) with a compound of formula (VI) is performed in a polar aprotic solvent. In some embodiments, the polar aprotic solvent is DMF.

In some embodiments, said reacting a compound of formula (V) with a compound of formula (VI) is performed at a temperature between 0° C. and 50° C., 0° C. and 30° C., 0° C. and 15° C., or 0° C. and 12° C.

In some embodiments, 2,4-bis(3-nitro-1H-pyrazol-1-yl)pyridine is separated from the compound of formula (IV) by recrystallization from a mixture of dichloromethane and methanol. In some embodiments, said recrystallization comprises dissolving the compound of formula (IV) in a mixture of dichloromethane and methanol, cooling the resulting solution, and then separating 2,4-bis(3-nitro-1H-pyrazol-1-yl)pyridine as a solid. In some embodiments, the dichloromethane and methanol are present in a ratio of between 1:2 and 1:3 by volume when the 2,4-bis(3-nitro-1H-pyrazol-1-yl)pyridine is separated.

In some embodiments, transforming the compound of formula (IV) to the compound of formula (I), or a pharmaceutically acceptable salt thereof, comprises:

reacting the compound of formula (IV) with H₂ in the presence of a suitable hydrogenation catalyst to afford a compound of formula (II)

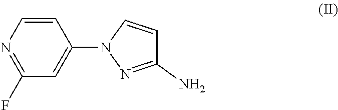 [SMILES: Nc1ccn(-c2ccnc(F)c2)n1]

and transforming the compound of formula (II) to the compound of formula (I), or a pharmaceutically acceptable salt thereof.

In some embodiments, the hydrogenation catalyst is a heterogeneous platinum catalyst. In some embodiments, the heterogeneous platinum catalyst is platinum on carbon.

In some embodiments, said reacting a compound of formula (IV) with H₂ is performed in a mixture of tetrahydrofuran and methanol.

In some embodiments, said H₂ is introduced in the form of H₂ gas.

In some embodiments, the compound of formula (II) is purified by recrystallization from a mixture of isopropyl alcohol and water. In some embodiments, said recrystallization comprises dissolving the compound of formula (II) in isopropyl alcohol, adding water to the resulting solution, and then separating the compound of formula (II) as a solid. In some embodiments, isopropyl alcohol and water are present in a ratio of between about 1:1 and about 3:1 by volume when the compound of formula (II) is separated.

In some embodiments, transforming the compound of formula (II) to the compound of formula (I), or a pharmaceutically acceptable salt thereof, comprises:

reacting the compound of formula (II) with a compound of formula (III)

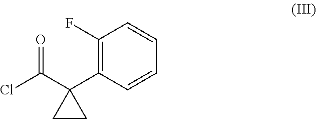 [SMILES: O=C(Cl)C1(c2ccccc2F)CC1]

in the presence of a suitable base to afford the compound of formula (I); and

optionally reacting the compound of formula (I) with a suitable acid to afford a pharmaceutically acceptable salt of the compound of formula (I).

In some embodiments, the base is a trialkylamine. In some embodiments, the trialkylamine is triethylamine.

In some embodiments, said reacting a compound of formula (II) with a compound of formula (III) is performed in tetrahydrofuran.

In some embodiments, the compound of formula (I) is purified by recrystallization from a mixture of isopropyl alcohol and water. In some embodiments, said recrystallization comprises dissolving the compound of formula (I) in isopropyl alcohol, adding water to the resulting solution, and then separating the compound of formula (I) as a solid. In some embodiments, isopropyl alcohol and water are present in a ratio of between about 2:1 and about 1:2 by volume when the compound of formula (I) is separated. In some embodiments, the compound of formula (I) purified by recrystallization contains no more than 5 wt. %, 4 wt. %, 3 wt. %, 2 wt. %, 1 wt. %, 5000 ppm, 4000 ppm, 3000 ppm, 2000 ppm, 1000 ppm, 500 ppm, 250 ppm, 100 ppm, 50 ppm, 25 ppm, or 20 ppm of the compound of formula (II).

In another aspect, the disclosure relates to a process for preparing a compound of formula (I)

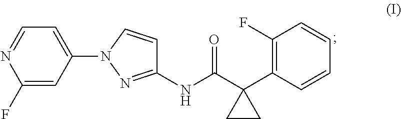 [SMILES: O=C(Nc1ccn(-c2ccnc(F)c2)n1)C1(c2ccccc2F)CC1]

comprising:

reacting a compound of formula (V)

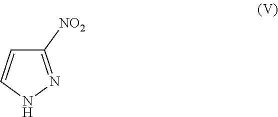

with a compound of formula (VI)

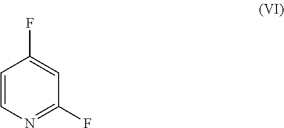

in the presence of a suitable base to afford a compound of formula (IV)

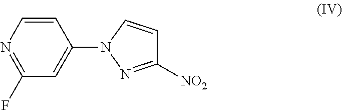 [SMILES: O=[N+]([O-])c1ccn(-c2ccnc(F)c2)n1]

and transforming the compound of formula (IV) to the compound of formula (I).

In some embodiments, the base is an inorganic phosphate or carbonate. In some embodiments, the base is K₃PO₄.

In some embodiments, said reacting a compound of formula (V) with a compound of formula (VI) is performed in a polar aprotic solvent. In some embodiments, the polar aprotic solvent is DMF.

In some embodiments, said reacting a compound of formula (V) with a compound of formula (VI) is performed at a temperature between 0° C. and 50° C., 0° C. and 30° C., 0° C. and 15° C., or 0° C. and 12° C.

In some embodiments, 2,4-bis(3-nitro-1H-pyrazol-1-yl)pyridine is separated from the compound of formula (IV) by recrystallization from a mixture of dichloromethane and methanol. In some embodiments, said recrystallization comprises dissolving the compound of formula (IV) in a mixture of dichloromethane and methanol, cooling the resulting solution, and then separating 2,4-bis(3-nitro-1H-pyrazol-1-yl)pyridine as a solid. In some embodiments, the dichloromethane and methanol are present in a ratio of between 1:2 and 1:3 by volume when the 2,4-bis(3-nitro-1H-pyrazol-1-yl)pyridine is separated.

In some embodiments, transforming the compound of formula (IV) to the compound of formula (I) comprises:

reacting the compound of formula (IV) with H₂ in the presence of a suitable hydrogenation catalyst to afford a compound of formula (II)

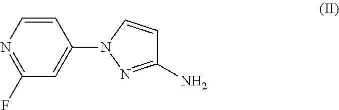 [SMILES: Nc1ccn(-c2ccnc(F)c2)n1]

and transforming the compound of formula (II) to the compound of formula (I).

In some embodiments, the hydrogenation catalyst is a heterogeneous platinum catalyst. In some embodiments, the heterogeneous platinum catalyst is platinum on carbon.

In some embodiments, said reacting a compound of formula (IV) with H₂ is performed in a mixture of tetrahydrofuran and methanol.

In some embodiments, said H₂ is introduced in the form of H₂ gas.

In some embodiments, the compound of formula (II) is purified by recrystallization from a mixture of isopropyl alcohol and water. In some embodiments, said recrystallization comprises dissolving the compound of formula (II) in isopropyl alcohol, adding water to the resulting solution, and then separating the compound of formula (II) as a solid. In some embodiments, isopropyl alcohol and water are present in a ratio of between about 1:1 and about 3:1 by volume when the compound of formula (II) is separated.

In some embodiments, transforming the compound of formula (II) to the compound of formula (I) comprises:

reacting the compound of formula (II) with a compound of formula (III)

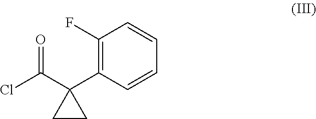 [SMILES: O=C(Cl)C1(c2ccccc2F)CC1]

in the presence of a suitable base to afford the compound of formula (I).

In some embodiments, the base is a trialkylamine. In some embodiments, the trialkylamine is triethylamine.

In some embodiments, said reacting a compound of formula (II) with a compound of formula (III) is performed in tetrahydrofuran.

In some embodiments, the compound of formula (I) is purified by recrystallization from a mixture of isopropyl alcohol and water. In some embodiments, said recrystallization comprises dissolving the compound of formula (I) in isopropyl alcohol, adding water to the resulting solution, and then separating the compound of formula (I) as a solid. In some embodiments, isopropyl alcohol and water are present in a ratio of between about 2:1 and about 1:2 by volume when the compound of formula (I) is separated. In some embodiments, the compound of formula (I) purified by recrystallization contains no more than 5 wt. %, 4 wt. %, 3 wt. %, 2 wt. %, 1 wt. %, 5000 ppm, 4000 ppm, 3000 ppm, 2000 ppm, 1000 ppm, 500 ppm, 250 ppm, 100 ppm, 50 ppm, 25 ppm, or 20 ppm of the compound of formula (II).

In some embodiments, the disclosure relates to a compound of formula (II)

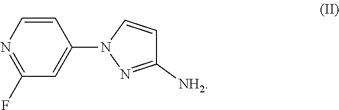 [SMILES: Nc1ccn(-c2ccnc(F)c2)n1]

In some embodiments, the disclosure relates to a compound of formula (III)

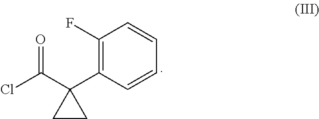 [SMILES: O=C(Cl)C1(c2ccccc2F)CC1]

In some embodiments, the disclosure relates to a compound of formula (IV)

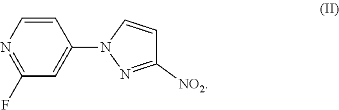 [SMILES: O=[N+]([O-])c1ccn(-c2ccnc(F)c2)n1]

### Definitions

For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compound” refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form or in a mixture (e.g., solution, suspension, or colloid) with one or more other substances.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

In some embodiments, the compounds disclosed and claimed herein, and pharmaceutically acceptable salts thereof, include each constituent atom at approximately the natural abundance isotopic composition of the specified element.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compound of formula (I) include those derived from suitable inorganic and organic acids. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

As used herein, the term “reacting,” when referring to two or more chemical species, means causing or allowing the chemical species to come into contact with one another under conditions suitable for a chemical reaction between the chemical species to occur.

As used herein, the term “trialkylamine” refers to a compound of the formula NR₃, wherein each R is independently C₁-C₆ alkyl, or wherein two R groups together with the nitrogen atom form a 5- or 6-membered heterocyclyl ring. Examples of trialkylamines include trimethylamine, diisopropylethylamine, N-methylmorpholine, and the like.

As used herein, the term “separating,” refers to the removal of some or all of one chemical component of a mixture from the remaining components of the mixture. For example, when a solid chemical component of a mixture is removed from one or more liquid components of the mixture, the solid component may be removed by means such as filtration, decantation, and the like.

As used herein, the term “hydrogenation catalyst” refers to those catalysts that are known to those skilled in the art to be effecting catalysts for the hydrogenation of organic molecules. Examples of suitable hydrogenation catalysts include heterogenous platinum catalysts (e.g., platinum on carbon, PtO₂.H₂O, and the like), heterogeneous palladium catalysts (e.g., palladium on carbon, palladium hydroxide on carbon (“Pearlman's Catalyst”), and the like), and heterogeneous nickel catalysts (e.g., Raney-Ni).

As used herein, the term “introduced,” when referring to the introduction of a compound or reagent to a chemical reaction, refers to the act of adding the compound or reagent to the reaction mixture. For example, where a particular compound or reagent is said to be “introduced” to a reaction mixture, it will be understood that the compound or reagent in question is not generated in situ.

As used herein, the term “inorganic phosphate” refers to a salt of the formula M₃PO₄, wherein M is a monovalent cation (e.g., Na⁺, K⁺, etc.). The term includes such salts in anhydrous, hydrated, and solvated forms. Examples of inorganic phosphates include Na₃PO₄, K₃PO₄, and the like.

As used herein, the term “inorganic carbonate” refers to a salt of the formula M₂CO₃ or DCO₃, wherein M is a monovalent cation (e.g., Na⁺, K⁺, etc.), and D is a divalent cation (e.g., Ca²⁺, Mg²⁺, etc.). The term includes such salts in anhydrous, hydrated, and solvated forms. Examples of inorganic carbonates include Na₂CO₃, Cs₂CO₃, CaCO₃, and the like.

As used herein, the term “transforming,” when referring to transforming one compound into another compound, means that the first compound is transformed into the second compound by any feasible reaction or series of reactions.

### Enumerated Embodiments

In some embodiments, provided are:

1. A process for preparing a compound of formula (I)

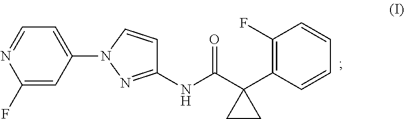 [SMILES: O=C(Nc1ccn(-c2ccnc(F)c2)n1)C1(c2ccccc2F)CC1]

or a pharmaceutically acceptable salt thereof, comprising:

reacting a compound of formula (II)

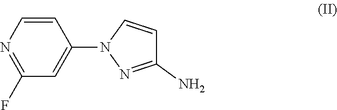 [SMILES: Nc1ccn(-c2ccnc(F)c2)n1]

with a compound of formula (III)

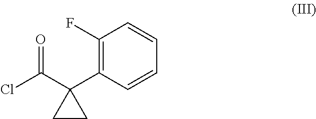 [SMILES: O=C(Cl)C1(c2ccccc2F)CC1]

in the presence of a suitable base to afford the compound of formula (I); and

optionally reacting the compound of formula (I) with a suitable acid to afford a pharmaceutically acceptable salt of the compound of formula (I).

2. The process of embodiment 1, wherein the base is a trialkylamine.
3. The process of embodiment 2, wherein the trialkylamine is triethylamine.
4. The process of any one of embodiments 1 to 3, wherein said reacting a compound of formula (II) with a compound of formula (III) is performed in tetrahydrofuran.
5. The process of any one of embodiments 1 to 4, wherein the compound of formula (I) is purified by recrystallization from a mixture of isopropyl alcohol and water.
6. The process of embodiment 5, wherein said recrystallization comprises dissolving the compound of formula (I) in isopropyl alcohol, adding water to the resulting solution, and then separating the compound of formula (I) as a solid.
7. The process of embodiment 6, wherein isopropyl alcohol and water are present in a ratio of between about 2:1 and about 1:2 by volume when the compound of formula (I) is separated.
8. The process of any one of embodiments 5 to 7, wherein the compound of formula (I) purified by recrystallization contains no more than 1 wt. % of the compound of formula (II).
9. The process of any one of embodiments 1 to 8, wherein the compound of formula (II) is obtained by reacting a compound of formula (IV)

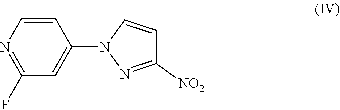 [SMILES: O=[N+]([O-])c1ccn(-c2ccnc(F)c2)n1]

with H₂ in the presence of a suitable hydrogenation catalyst to afford the compound of formula (II).
10. The process of embodiment 9, wherein the hydrogenation catalyst is a heterogeneous platinum catalyst.
11. The process of embodiment 10, wherein the heterogeneous platinum catalyst is platinum on carbon.
12. The process of any one of embodiments 9 to 11, wherein said reacting a compound of formula (IV) with H₂ is performed in a mixture of tetrahydrofuran and methanol.
13. The process of any one of embodiments 9 to 12, wherein said H₂ is introduced in the form of H₂ gas.
14. The process of any one of embodiments 9 to 13, wherein the compound of formula (II) is purified by recrystallization from a mixture of isopropyl alcohol and water.
15. The process of embodiment 14, wherein said recrystallization comprises dissolving the compound of formula (II) in isopropyl alcohol, adding water to the resulting solution, and then separating the compound of formula (II) as a solid.
16. The process of embodiment 15, wherein isopropyl alcohol and water are present in a ratio of between about 1:1 and about 3:1 by volume when the compound of formula (II) is separated.
17. The process of any one of embodiments 9 to 16, wherein the compound of formula (IV) is obtained by reacting a compound of formula (V)

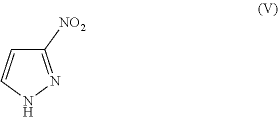

with a compound of formula (VI)

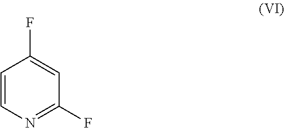

in the presence of a suitable base to afford the compound of formula (IV).
18. The process of embodiment 17, wherein the base is an inorganic phosphate or carbonate.
19. The process of embodiment 18, wherein the base is K₃PO₄.
20. The process of any one of embodiments 17 to 19, wherein said reacting a compound of formula (V) with a compound of formula (VI) is performed in DMF.
21. The process of any one of embodiments 17 to 20, wherein said reacting a compound of formula (V) with a compound of formula (VI) is performed at a temperature between 0° C. and 15° C.
22. The process of any one of embodiments 17 to 21, further comprising separating 2,4-bis(3-nitro-1H-pyrazol-1-yl)pyridine from the compound of formula (IV) by recrystallization from a mixture of dichloromethane and methanol.
23. The process of embodiment 22, wherein said recrystallization comprises dissolving the compound of formula (IV) in a mixture of dichloromethane and methanol, cooling the resulting solution, and then separating 2,4-bis(3-nitro-1H-pyrazol-1-yl)pyridine as a solid.
24. The process of embodiment 23, wherein the dichloromethane and methanol are present in a ratio of between 1:2 and 1:3 by volume when the 2,4-bis(3-nitro-1H-pyrazol-1-yl)pyridine is separated.
25. A process for preparing a compound of formula (I)

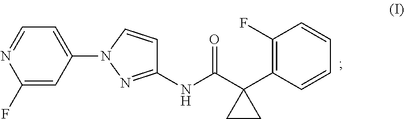 [SMILES: O=C(Nc1ccn(-c2ccnc(F)c2)n1)C1(c2ccccc2F)CC1]

or a pharmaceutically acceptable salt thereof, comprising:

reacting a compound of formula (V)

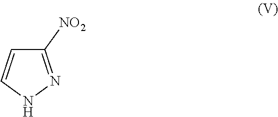

with a compound of formula (VI)

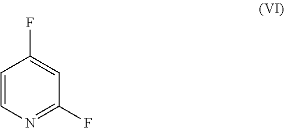

in the presence of a suitable base to afford a compound of formula (IV)

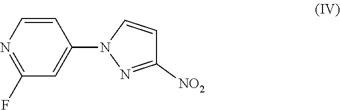 [SMILES: O=[N+]([O-])c1ccn(-c2ccnc(F)c2)n1]

and transforming the compound of formula (IV) to the compound of formula (I), or a pharmaceutically acceptable salt thereof.
26. The process of embodiment 25, wherein the base is an inorganic phosphate or carbonate.
27. The process of embodiment 26, wherein the base is K₃PO₄.
28. The process of any one of embodiments 25 to 27, wherein said reacting a compound of formula (V) with a compound of formula (VI) is performed in DMF.
29. The process of any one of embodiments 25 to 28, wherein said reacting a compound of formula (V) with a compound of formula (VI) is performed at a temperature between 0° C. and 15° C.
30. The process of any one of embodiments 25 to 29, further comprising separating 2,4-bis(3-nitro-1H-pyrazol-1-yl)pyridine from the compound of formula (IV) by recrystallization from a mixture of dichloromethane and methanol.
31. The process of embodiment 30, wherein said recrystallization comprises dissolving the compound of formula (IV) in a mixture of dichloromethane and methanol, cooling the resulting solution, and then separating 2,4-bis(3-nitro-1H-pyrazol-1-yl)pyridine as a solid.
32. The process of embodiment 31, wherein the dichloromethane and methanol are present in a ratio of between 1:2 and 1:3 by volume when the 2,4-bis(3-nitro-1H-pyrazol-1-yl)pyridine is separated.
33. The process of any one of embodiments 25 to 32, wherein said transforming the compound of formula (IV) to the compound of formula (I), or a pharmaceutically acceptable salt thereof, comprises:

reacting the compound of formula (IV) with H₂ in the presence of a suitable hydrogenation catalyst to afford a compound of formula (II)

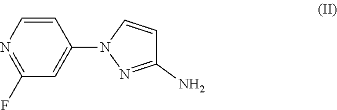 [SMILES: Nc1ccn(-c2ccnc(F)c2)n1]

and transforming the compound of formula (II) to the compound of formula (I), or a pharmaceutically acceptable salt thereof.
34. The process of embodiment 33, wherein the hydrogenation catalyst is a heterogeneous platinum catalyst.
35. The process of embodiment 34, wherein the heterogeneous platinum catalyst is platinum on carbon.
36. The process of any one of embodiments 33 to 35, wherein said reacting a compound of formula (IV) with H₂ is performed in a mixture of tetrahydrofuran and methanol.
37. The process of any one of embodiments 33 to 36, wherein H₂ is introduced in the form of H₂ gas.
38. The process of any one of embodiments 33 to 37, wherein the compound of formula (II) is purified by recrystallization from a mixture of isopropyl alcohol and water.
39. The process of embodiment 38, wherein said recrystallization comprises dissolving the compound of formula (II) in isopropyl alcohol, adding water to the resulting solution, and then separating the compound of formula (II) as a solid.
40. The process of embodiment 39, wherein isopropyl alcohol and water are present in a ratio of between about 1:1 and about 3:1 by volume when the compound of formula (II) is separated.
41. The process of any one of embodiments 33 to 40, wherein said transforming the compound of formula (II) to the compound of formula (I), or a pharmaceutically acceptable salt thereof, comprises:

reacting the compound of formula (II) with a compound of formula (III)

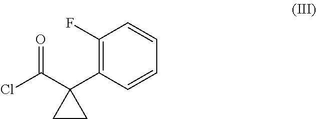 [SMILES: O=C(Cl)C1(c2ccccc2F)CC1]

in the presence of a suitable base to afford the compound of formula (I); and

optionally reacting the compound of formula (I) with a suitable acid to afford a pharmaceutically acceptable salt of the compound of formula (I).

42. The process of embodiment 41, wherein the base is a trialkylamine.
43. The process of embodiment 42, wherein the trialkylamine is triethylamine.
44. The process of any one of embodiments 41 to 43, wherein said reacting a compound of formula (II) with a compound of formula (III) is performed in tetrahydrofuran.
45. The process of any one of embodiments 41 to 44, wherein the compound of formula (I) is purified by recrystallization from a mixture of isopropyl alcohol and water.
46. The process of embodiment 45, wherein said recrystallization comprises dissolving the compound of formula (I) in isopropyl alcohol, adding water to the resulting solution, and then separating the compound of formula (I) as a solid.
47. The process of embodiment 46, wherein isopropyl alcohol and water are present in a ratio of between about 2:1 and about 1:2 by volume when the compound of formula (I) is separated.
48. The process of any one of embodiments 45-47, wherein the compound of formula (I) purified by recrystallization contains not more than 5 wt. %, 4 wt. %, 3 wt. %, 2 wt. %, 1 wt. %, 5000 ppm, 4000 ppm, 3000 ppm, 2000 ppm, 1000 ppm, 500 ppm, 250 ppm, 100 ppm, 50 ppm, 25 ppm, or 20 ppm of the compound of formula (II).
49. A process for preparing a compound of formula (I)

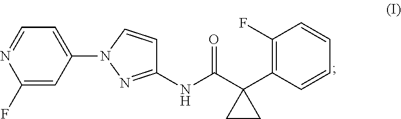 [SMILES: O=C(Nc1ccn(-c2ccnc(F)c2)n1)C1(c2ccccc2F)CC1]

comprising:

reacting a compound of formula (II)

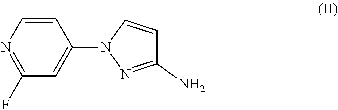 [SMILES: Nc1ccn(-c2ccnc(F)c2)n1]

with a compound of formula (III)

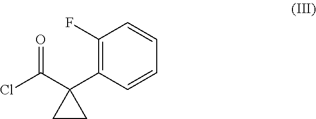 [SMILES: O=C(Cl)C1(c2ccccc2F)CC1]

in the presence of a suitable base to afford the compound of formula (I).
50. The process of embodiment 49, wherein the base is a trialkylamine.
51. The process of embodiment 50, wherein the trialkylamine is triethylamine.
52. The process of any one of embodiments 49 to 51, wherein said reacting a compound of formula (II) with a compound of formula (III) is performed in tetrahydrofuran.
53. The process of any one of embodiments 49 to 52, wherein the compound of formula (I) is purified by recrystallization from a mixture of isopropyl alcohol and water.
54. The process of embodiment 53, wherein said recrystallization comprises dissolving the compound of formula (I) in isopropyl alcohol, adding water to the resulting solution, and then separating the compound of formula (I) as a solid.
55. The process of embodiment 54, wherein isopropyl alcohol and water are present in a ratio of between about 2:1 and about 1:2 by volume when the compound of formula (I) is separated.
56. The process of any one of embodiments 53 to 55, wherein the compound of formula (I) purified by recrystallization contains no more than 1 wt. % of the compound of formula (II).
57. The process of any one of embodiments 49 to 56, wherein the compound of formula (II) is obtained by reacting a compound of formula (IV)

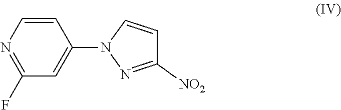 [SMILES: O=[N+]([O-])c1ccn(-c2ccnc(F)c2)n1]

with H₂ in the presence of a suitable hydrogenation catalyst to afford the compound of formula (II).
58. The process of embodiment 57, wherein the hydrogenation catalyst is a heterogeneous platinum catalyst.
59. The process of embodiment 58, wherein the heterogeneous platinum catalyst is platinum on carbon.
60. The process of any one of embodiments 57 to 59, wherein said reacting a compound of formula (IV) with H₂ is performed in a mixture of tetrahydrofuran and methanol.
61. The process of any one of embodiments 57 to 60, wherein said H₂ is introduced in the form of H₂ gas.
62. The process of any one of embodiments 57 to 61, wherein the compound of formula (II) is purified by recrystallization from a mixture of isopropyl alcohol and water.
63. The process of embodiment 62, wherein said recrystallization comprises dissolving the compound of formula (II) in isopropyl alcohol, adding water to the resulting solution, and then separating the compound of formula (II) as a solid.
64. The process of embodiment 63, wherein isopropyl alcohol and water are present in a ratio of between about 1:1 and about 3:1 by volume when the compound of formula (II) is separated.
65. The process of any one of embodiments 57 to 64, wherein the compound of formula (IV) is obtained by reacting a compound of formula (V)

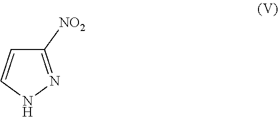

with a compound of formula (VI)

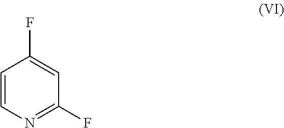

in the presence of a suitable base to afford the compound of formula (IV).
66. The process of embodiment 65, wherein the base is an inorganic phosphate or carbonate.
67. The process of embodiment 66, wherein the base is K₃PO₄.
68. The process of any one of embodiments 65 to 67, wherein said reacting a compound of formula (V) with a compound of formula (VI) is performed in DMF.
69. The process of any one of embodiments 65 to 68, wherein said reacting a compound of formula (V) with a compound of formula (VI) is performed at a temperature between 0° C. and 15° C.
70. The process of any one of embodiments 65 to 69, further comprising separating 2,4-bis(3-nitro-1H-pyrazol-1-yl)pyridine from the compound of formula (IV) by recrystallization from a mixture of dichloromethane and methanol.
71. The process of embodiment 70, wherein said recrystallization comprises dissolving the compound of formula (IV) in a mixture of dichloromethane and methanol, cooling the resulting solution, and then separating 2,4-bis(3-nitro-1H-pyrazol-1-yl)pyridine as a solid.
72. The process of embodiment 71, wherein the dichloromethane and methanol are present in a ratio of between 1:2 and 1:3 by volume when the 2,4-bis(3-nitro-1H-pyrazol-1-yl)pyridine is separated.
73. A process for preparing a compound of formula (I)

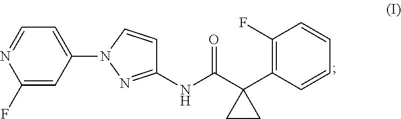 [SMILES: O=C(Nc1ccn(-c2ccnc(F)c2)n1)C1(c2ccccc2F)CC1]

comprising:

reacting a compound of formula (V)

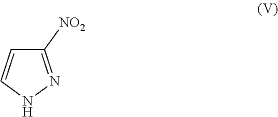

with a compound of formula (VI)

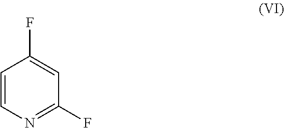

in the presence of a suitable base to afford a compound of formula (IV)

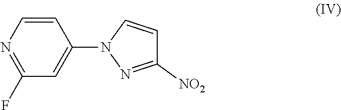 [SMILES: O=[N+]([O-])c1ccn(-c2ccnc(F)c2)n1]

and transforming the compound of formula (IV) to the compound of formula (I).
74. The process of embodiment 73, wherein the base is an inorganic phosphate or carbonate.
75. The process of embodiment 74, wherein the base is K₃PO₄.
76. The process of any one of embodiments 73 to 75, wherein said reacting a compound of formula (V) with a compound of formula (VI) is performed in DMF.
77. The process of any one of embodiments 73 to 76, wherein said reacting a compound of formula (V) with a compound of formula (VI) is performed at a temperature between 0° C. and 15° C.
78. The process of any one of embodiments 73 to 77, further comprising separating 2,4-bis(3-nitro-1H-pyrazol-1-yl)pyridine from the compound of formula (IV) by recrystallization from a mixture of dichloromethane and methanol.
79. The process of embodiment 78, wherein said recrystallization comprises dissolving the compound of formula (IV) in a mixture of dichloromethane and methanol, cooling the resulting solution, and then separating 2,4-bis(3-nitro-1H-pyrazol-1-yl)pyridine as a solid.
80. The process of embodiment 79, wherein the dichloromethane and methanol are present in a ratio of between 1:2 and 1:3 by volume when the 2,4-bis(3-nitro-1H-pyrazol-1-yl)pyridine is separated.
81. The process of any one of embodiments 73 to 80, wherein said transforming the compound of formula (IV) to the compound of formula (I) comprises:

reacting the compound of formula (IV) with H₂ in the presence of a suitable hydrogenation catalyst to afford a compound of formula (II)

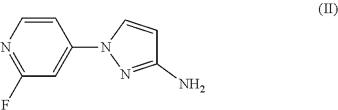 [SMILES: Nc1ccn(-c2ccnc(F)c2)n1]

and transforming the compound of formula (II) to the compound of formula (I).
82. The process of embodiment 81, wherein the hydrogenation catalyst is a heterogeneous platinum catalyst.
83. The process of embodiment 82, wherein the heterogeneous platinum catalyst is platinum on carbon.
84. The process of any one of embodiments 81 to 83, wherein said reacting a compound of formula (IV) with H₂ is performed in a mixture of tetrahydrofuran and methanol.
85. The process of any one of embodiments 81 to 84, wherein H₂ is introduced in the form of H₂ gas.
86. The process of any one of embodiments 81 to 85, wherein the compound of formula (II) is purified by recrystallization from a mixture of isopropyl alcohol and water.
87. The process of embodiment 86, wherein said recrystallization comprises dissolving the compound of formula (II) in isopropyl alcohol, adding water to the resulting solution, and then separating the compound of formula (II) as a solid.
88. The process of embodiment 87, wherein isopropyl alcohol and water are present in a ratio of between about 1:1 and about 3:1 by volume when the compound of formula (II) is separated.
89. The process of any one of embodiments 81 to 88, wherein said transforming the compound of formula (II) to the compound of formula (I) comprises:

reacting the compound of formula (II) with a compound of formula (III)

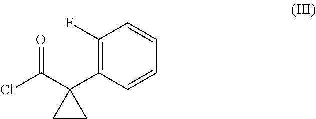 [SMILES: O=C(Cl)C1(c2ccccc2F)CC1]

in the presence of a suitable base to afford the compound of formula (I).
90. The process of embodiment 89, wherein the base is a trialkylamine.
91. The process of embodiment 90, wherein the trialkylamine is triethylamine.
92. The process of any one of embodiments 89 to 91, wherein said reacting a compound of formula (II) with a compound of formula (III) is performed in tetrahydrofuran.
93. The process of any one of embodiments 89 to 92, wherein the compound of formula (I) is purified by recrystallization from a mixture of isopropyl alcohol and water.
94. The process of embodiment 93, wherein said recrystallization comprises dissolving the compound of formula (I) in isopropyl alcohol, adding water to the resulting solution, and then separating the compound of formula (I) as a solid.
95. The process of embodiment 94, wherein isopropyl alcohol and water are present in a ratio of between about 2:1 and about 1:2 by volume when the compound of formula (I) is separated.
96. The process of any one of embodiments 93-95, wherein the compound of formula (I) purified by recrystallization contains not more than 5 wt. %, 4 wt. %, 3 wt. %, 2 wt. %, 1 wt. %, 5000 ppm, 4000 ppm, 3000 ppm, 2000 ppm, 1000 ppm, 500 ppm, 250 ppm, 100 ppm, 50 ppm, 25 ppm, or 20 ppm of the compound of formula (II).
97. A compound of formula (II)

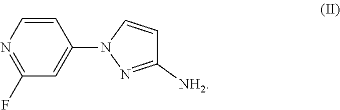 [SMILES: Nc1ccn(-c2ccnc(F)c2)n1]

98. A compound of formula (III)

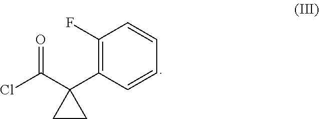 [SMILES: O=C(Cl)C1(c2ccccc2F)CC1]

99. A compound of formula (IV)

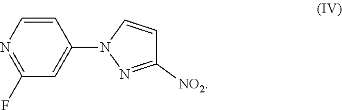 [SMILES: O=[N+]([O-])c1ccn(-c2ccnc(F)c2)n1]

### EXAMPLES

### Example 1

### Preparation of 1-(2-fluorophenyl)-N-(1-(2-fluoropyridin-4-yl)-1H-pyrazol-3-yl)cyclopropane-1-carboxamide

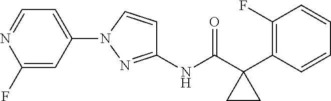 [SMILES: O=C(Nc1ccn(-c2ccnc(F)c2)n1)C1(c2ccccc2F)CC1]

### Step 1: Preparation of 2-fluoro-4-(3-nitro-1H-pyrazol-1-yl)pyridine

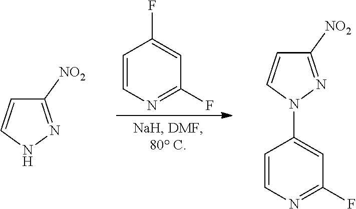 [SMILES: O=[N+]([O-])c1cc[nH]n1 ; Fc1ccnc(F)c1 ; O=[N+]([O-])c1ccn(-c2ccnc(F)c2)n1]

To a 0° C. solution of 3-nitro-1H-pyrazole (250.0 g, 2.17 mol, 1.0 eq) in anhydrous DMF (2.5 L; 10.2 vol eq) under nitrogen was added NaH (95.42 g of 60% w/w, 2.39 mol, 1.1 eq) in batches over 30 min while maintaining temperature below 8° C. The mixture was stirred for 1 h then 2,4-difluoropyridine (300 mL, 3.29 mol, 1.5 eq) was added, and the reaction was warmed to room temperature and stirred for approximately 16 hours (h). The reaction mixture was diluted with water (12.5 L) and stirred vigorously for 1 h. The off-white solid was collected by vacuum filtration. The solid was re-suspended in water (2 L) and filtered, and this step was repeated once further. The product was dried under vacuum, then suspended in heptane (4 L), stirred 3 h at room temperature, and filtered. The solid was washed with two further portions of heptane (2 L each) and dried under vacuum to provide 2-fluoro-4-(3-nitro-1H-pyrazol-1-yl)pyridine (426.3 g of 92% purity, 87% yield). 1H NMR (400 MHz, DMSO-d6) δ 9.01 (d, J=2.8 Hz, 1H), 8.45 (d, J=5.7 Hz, 1H), 7.95 (ddd, J=5.7, 1.9, 1.2 Hz, 1H), 7.81 (t, J=1.4 Hz, 1H), 7.46 (d, J=2.8 Hz, 1H) ppm. ESI-MS m/z calc. 208.04, found 209.01 (M+1).

### Step 2: Preparation of 1-(2-fluoropyridin-4-yl)-1H-pyrazol-3-amine

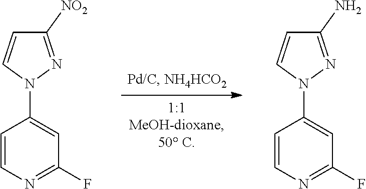 [SMILES: O=[N+]([O-])c1ccn(-c2ccnc(F)c2)n1 ; Nc1ccn(-c2ccnc(F)c2)n1]

A mixture of 2-fluoro-4-(3-nitropyrazol-1-yl)pyridine (200.0 g, 893.6 mol, 1.0 eq), 10% Pd/C (18.60 g of 10% w/w, 17.48 mmol, 0.02 eq), ammonium formate (572.95 g, 8.814 mol, 10 eq), methanol (500 mL; 2.7 vol eq), and dioxane (1.0 L; 5.4 vol eq) was stirred at 50° C. until starting materials were consumed, which was about 2.5 h. The reaction mixture was hot-filtered through Celite, and the filter cake was washed with dioxane (500 mL) and methanol (250 mL). The combined filtrate was concentrated to a white solid. The solid was suspended in water (3 L), stirred overnight (about 16 h), and filtered. Water (1 L) was added, mixture stirred, filtered, and dried on vac line for about 6 h. The product was dried at 55° C. under vacuum overnight to provide 1-(2-fluoropyridin-4-yl)-1H-pyrazol-3-amine (145.0 g, 89% yield). 1H NMR (400 MHz, DMSO-d6) δ 8.35 (d, J=2.8 Hz, 1H), 8.14 (d, J=5.8 Hz, 1H), 7.56 (dt, J=5.7, 1.7 Hz, 1H), 7.28 (d, J=1.8 Hz, 1H), 5.91 (d, J=2.8 Hz, 1H), 5.47 (s, 2H) ppm. ESI-MS m/z calc. 178.07, found 178.98 (M+1).

### Step 3: Preparation of 1-(2-fluorophenyl)cyclopropanecarbonyl Chloride

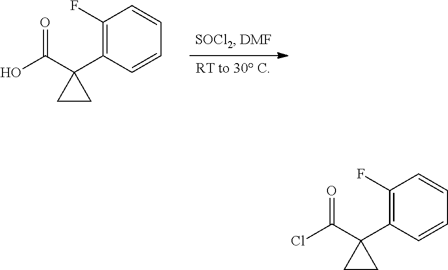 [SMILES: O=C(O)C1(c2ccccc2F)CC1 ; O=C(Cl)C1(c2ccccc2F)CC1]

To a solution/suspension or 1-(2-fluorophenyl)cyclopropane-1-carboxylic acid (266 g, 1.46 mol, 1.3 eq) in thionyl chloride (SOCl₂; 295 mL, 4.04 mol, 3.6 eq) at room temperature was added DMF (800 μL, 10.33 mmol, 0.01 eq). The resultant solution was stirred 1 hour (h) at room temperature and 3 h at 30° C. The solvent was removed in vacuo, and excess thionyl chloride and HCl were removed by azeotrope with toluene (100 mL). 1-(2-fluorophenyl)cyclopropanecarbonyl chloride (290 g, 100%) was obtained as a clear yellow oil. 1H NMR (400 MHz, CDCl3) δ 7.44-7.24 (m, 2H), 7.24-7.05 (m, 2H), 2.11-1.96 (m, 2H), 1.59-1.43 (m, 2H) ppm. ESI-MS m/z calc. 198.02, found 199.63 (M+1)+.

### Step 4: Preparation of 1-(2-fluorophenyl)-N-(1-(2-fluoropyridin-4-yl)-1H-pyrazol-3-yl)cyclopropane-1-carboxamide

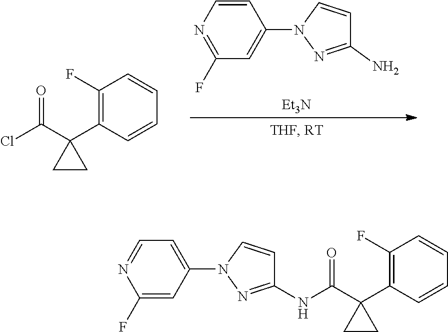

To a 0° C. suspension of 1-(2-fluoropyridin-4-yl)-1H-pyrazol-3-amine (200 g, 1.12 mol, 1.0 eq) and triethylamine (Et₃N; 391 mL, 2.81 mol, 2.5 eq) in THF (1.6 L) was added 1-(2-fluorophenyl)cyclopropanecarbonyl chloride (290 g, 1.46 mol, 1.3 eq) slowly over 1 h so as to maintain the reaction temperature below 8° C. The reaction mixture was stirred a further for 1 h in the ice-bath then warmed to room temperature for approximately 16 h. After water (200 mL) was added and stirred for about 20 minutes, the THF was removed in vacuo. The resultant mixture was partitioned between ethyl acetate (6.5 L) and aqueous 5% Na₂CO₃ (3 L). The layers were separated, and the organic layer was washed with aqueous 5% Na₂CO₃ (3 L), dried and concentrated. The crude residue was purified by silica gel chromatography (linear gradient of 0-100% ethyl acetate/heptane). Relevant fractions were combined and concentrated to provide the desired product, which was re-suspended in heptane (4 L) and circulated on a rotary evaporator at atmospheric pressure for approximately 16 h. The product was collected by filtration, washed twice with heptane, and dried in vacuo to provide 1-(2-fluorophenyl)-N-(1-(2-fluoropyridin-4-yl)-1H-pyrazol-3-yl)cyclopropane-1-carboxamide (300 g, 78% yield; white crystalline solid). 1H-NMR (400 MHz, DMSO-d6) δ 9.59 (s, 1H), 8.63 (d, J=2.8 Hz, 1H), 8.25 (d, J=5.7 Hz, 1H), 7.71 (dt, J=5.7, 1.5 Hz, 1H), 7.55-7.44 (m, 2H), 7.44-7.33 (m, 1H), 7.28-7.13 (m, 2H), 6.88 (d, J=2.8 Hz, 1H), 1.71-1.54 (m, 2H), 1.25-1.08 (m, 2H) ppm.

### Example 2

### Preparation of 1-(2-fluorophenyl)-N-(1-(2-fluoropyridin-4-yl)-1H-pyrazol-3-yl)cyclopropane-1-carboxamide

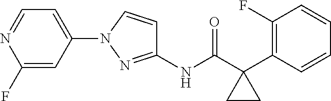 [SMILES: O=C(Nc1ccn(-c2ccnc(F)c2)n1)C1(c2ccccc2F)CC1]

### Step 1: Preparation of 2-fluoro-4-(3-nitro-1H-pyrazol-1-yl)pyridine

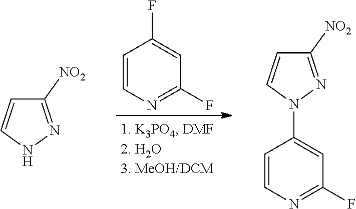 [SMILES: O=[N+]([O-])c1cc[nH]n1 ; Fc1ccnc(F)c1 ; O=[N+]([O-])c1ccn(-c2ccnc(F)c2)n1]

A reactor was charged with 3-nitro-1H-pyrazole (300 g, 2.67 mol, limiting reagent). Anhydrous DMF (2.4 L, 8 vol.) was added, and stirring was begun. The solution was cooled to 13° C., and K₃PO₄ (1.13 kg, 5.33 mol, 2 eq) was added. 2,4-difluoropyridine (613.9 g, 5.33 mol, 2 eq) was added to the reactor, and the reaction was stirred until complete. The reaction mixture was filtered, and the filtrate was transferred slowly into a reactor containing water (6 L, 20 vol.). The resulting slurry was stirred for 1 h. The slurry was then filtered, and the wet cake was washed with water and dried in a vacuum oven at 60° C. Crude 2-fluoro-4-(3-nitro-1H-pyrazol-1-yl)pyridine was isolated in 89% yield as an off white solid. The crude material contained between 7 and 9% (HPLC area percent) of 2,4-bis(3-nitro-1H-pyrazol-1-yl)pyridine, which was formed as a side product.

2-fluoro-4-(3-nitro-1H-pyrazol-1-yl)pyridine was separated from 2,4-bis(3-nitro-1H-pyrazol-1-yl)pyridine (formed as a side product) by recrystallization. A reactor was charged with crude 2-fluoro-4-(3-nitro-1H-pyrazol-1-yl)pyridine (944.1 g), dichloromethane (8.5 L, 9 vol.), and methanol (19.8 L, 21 vol.), and the agitation was set to 150 rpm. The slurry was stirred at 39° C. for about 4 h, and then the jacket temperature was ramped down to 20° C., and stirring was continued for 30 minutes. The reaction mixture was filtered, and the wet cake was rinsed with methanol (0.5 L, 0.6 vol.). The filtrate was concentrated, and the resulting slurry was filtered. The wet cake was rinsed with methanol and then dried in a vacuum oven at 50-55° C. with nitrogen bleed. 2-fluoro-4-(3-nitro-1H-pyrazol-1-yl)pyridine was isolated in 75% yield (708 g) as a white solid. The identity of the material obtained was confirmed by comparing its HPLC retention time to that of an authentic sample. The isolated material contained less than 2% (HPLC area percent) of 2,4-bis(3-nitro-1H-pyrazol-1-yl)pyridine.

### Step 2: Preparation of 1-(2-fluoropyridin-4-yl)-1H-pyrazol-3-amine

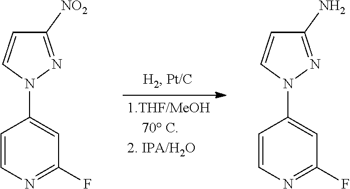 [SMILES: O=[N+]([O-])c1ccn(-c2ccnc(F)c2)n1 ; Nc1ccn(-c2ccnc(F)c2)n1]

2-fluoro-4-(3-nitro-1H-pyrazol-1-yl)pyridine (808 g, 3.88 mol, 1 eq), 3% platinum on carbon catalyst (66% wet) (37.9 g, 1.94 mol, 0.0005 eq), and 2:1 tetrahydrofuran:methanol (13.6 L, 17 vol.) were loaded into a jacketed hydrogenator. The hydrogenator was purged with nitrogen and was then purged with hydrogen. The hydrogen was charged to a pressure of 3.0 bar, and the jacket temperature was ramped to 50° C. over 1 hour. Stirring was maintained between about 800 and 1,000 RPM. The batch was stirred until complete conversion was achieved (˜10 hours). The batch was cooled to 30° C. and filtered over a Celite pad to remove the catalyst. The filter cake was washed with 2:1 tetrahydrofuran:methanol (1.76 L, 2 vol.), the tetrahydrofuran/methanol mother liquors were stripped to dry solid, and two chases of isopropyl alcohol (each 5 volumes) were performed to remove as much tetrahydrofuran as possible. The solids were then taken up in 8 volumes of isopropyl alcohol (6.5 L) and heated to 80° C. Once temperature was reached, 4 volumes of water (3.2 L) were added over 1 hour to afford a clear, yellow solution. The solution was cooled to 70° C. and was seeded with crystals of 1-(2-fluoropyridin-4-yl)-1H-pyrazol-3-amine (0.05 wt %, 4 g). Crystals were allowed to grow as the batch was cooled from 70° C. to 60° C. over 1 hour, and then another 12 volumes of water (9.7 L) were added over two hours. Once the water addition was complete, the batch was cooled from 60° C. to 20° C. over 5 hours and was then filtered and washed with 2 volumes of 2:1 water:isopropyl alcohol (2.4 mL). The solids were dried in an oven at 45° C. with a nitrogen sweep until a constant weight was obtained. 1-(2-fluoropyridin-4-yl)-1H-pyrazol-3-amine was obtained in 88% yield. The identity of the material obtained was confirmed by comparing its HPLC retention time to that of an authentic sample.

### Step 3: Preparation of 1-(2-fluorophenyl)cyclopropanecarbonyl Chloride

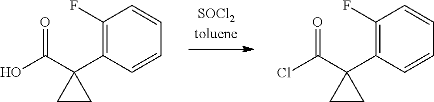 [SMILES: O=C(O)C1(c2ccccc2F)CC1 ; O=C(Cl)C1(c2ccccc2F)CC1]

A reactor was charged with 1-(2-fluorophenyl)cyclopropane-1-carboxylic acid (1750.6 g, 9.72 mol, limiting reagent), and toluene (3.5 L, 2 vol) was added. Thionyl chloride (1417 mL, 19.43 mol, 2 eq) was added to reactor, and the reaction was heated to 35-40° C. Upon completion of the reaction, toluene (7 L, 4 vol) was added to the reactor, and the reaction mixture was distilled to dryness to obtain 1-(2-fluorophenyl)cyclopropanecarbonyl chloride in 98% yield as a yellow oil. The identity of the material obtained was confirmed by quenching the acid chloride with methanol and comparing the HPLC retention time of the resulting material to that of an authentic sample of the expected methyl ester. The purity of the acid chloride was determined by ¹H NMR analysis.

### Step 4: Preparation of 1-(2-fluorophenyl)-N-(1-(2-fluoropyridin-4-yl)-1H-pyrazol-3-yl)cyclopropane-1-carboxamide

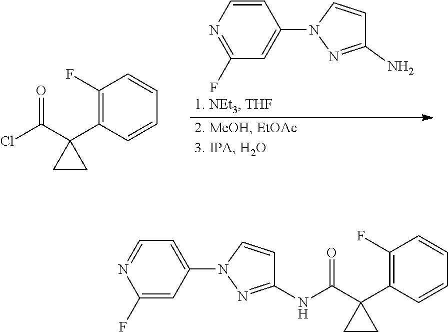

A reactor was charged with 1-(2-fluoropyridin-4-yl)-1H-pyrazol-3-amine (1499.9 g, 8.42 mol, limiting reagent) and tetrahydrofuran (15 L, 10 vol). Triethylamine (2.35 L, 16.84 mol, 2 eq) was added at 13° C. A solution of 1-(2-fluorophenyl)cyclopropanecarbonyl chloride (1672.4 g, 8.42 mol, 1.0 eq) in tetrahydrofuran (3.0 L, 2 vol) was added to the reactor, while maintaining a temperature of 13-18° C. Upon reaction completion, methanol (0.75 L, 0.5 vol) was added, and the mixture was stirred for no less than 30 minutes. Water (6 L, 4 vol) was added to the reactor at 14° C., and the mixture was allowed to warm up to ambient temperature. The reaction mixture was extracted with ethyl acetate (7.5 L, 5 vol), and the organic layer was washed with 1 N HCl (6.76 L, 4.5 vol), followed by water (6 L, 4 vol). The organic layer was concentrated, isopropyl alcohol (11.25 L, 7.5 vol.) was added, and the mixture was heated to 75° C. Water (3.8 L, 2.5 vol) was added to the reactor over 1 h, while maintaining a temperature greater than 70° C. Seed crystals of 1-(2-fluorophenyl)-N-(1-(2-fluoropyridin-4-yl)-1H-pyrazol-3-yl)cyclopropane-1-carboxamide (28.7 g, 0.08 mol, 0.01 eq) were added at 55° C., and the mixture was stirred for 30 minutes. Water (7.5 L, 5 vol) was added to the reactor at 50-55° C. over 5 h, and then the jacket was ramped down to 20° C. over 5 hours. Stirring was continued at 20° C. for 30 minutes, and then the batch was filtered and washed with 1:1 isopropyl alcohol:water (3.8 L). The wet cake was transferred to drying trays and dried in a vacuum oven at 45° C. with nitrogen bleed. 1-(2-fluorophenyl)-N-(1-(2-fluoropyridin-4-yl)-1H-pyrazol-3-yl)cyclopropane-1-carboxamide was obtained in 83.5% yield. The isolated material contained less than 20 ppm of 1-(2-fluoropyridin-4-yl)-1H-pyrazol-3-amine. The identity of the material obtained was confirmed by comparing its HPLC retention time to that of an authentic sample.

### Example 3

### Preparation of 1-(2-fluorophenyl)-N-(1-(2-fluoropyridin-4-yl)-1H-pyrazol-3-yl)cyclopropane-1-carboxamide by Continuous Flow Chemistry (Proof of Concept)

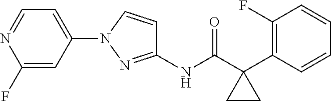 [SMILES: O=C(Nc1ccn(-c2ccnc(F)c2)n1)C1(c2ccccc2F)CC1]

Steps 1-4 of the preparation of 1-(2-fluorophenyl)-N-(1-(2-fluoropyridin-4-yl)-1H-pyrazol-3-yl)cyclopropane-1-carboxamide (I) were conducted individually in a continuous flow reactor to establish proof of concept for a continuous flow process.

### Step 1: Preparation of 2-fluoro-4-(3-nitro-1H-pyrazol-1-yl)pyridine

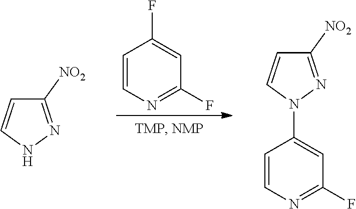 [SMILES: O=[N+]([O-])c1cc[nH]n1 ; Fc1ccnc(F)c1 ; O=[N+]([O-])c1ccn(-c2ccnc(F)c2)n1]

A flask was charged with 3-nitro-1H-pyrazole (5.00 g, 44.218 mmol, 1.000 equiv). N-methylpyrrolidone (20.20 g, 19.6 mL) was added with stirring to obtain a clear solution, and then 2,2,6,6-tetramethylpiperidine (6.87 g, 8.3 mL, 48.640 mmol, 1.100 equiv) was added. In a separate flask, 2,4-difluoropyridine (7.63 g, 66.328 mmol, 1.500 equiv) was dissolved in N-methylpyrrolidone (20.588 g, 19.98 mL). The two streams were combined in a Coreflow continuous reactor.

### Step 2: Preparation of 1-(2-fluoropyridin-4-yl)-1H-pyrazol-3-amine

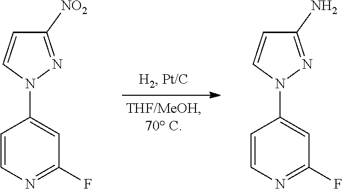 [SMILES: O=[N+]([O-])c1ccn(-c2ccnc(F)c2)n1 ; Nc1ccn(-c2ccnc(F)c2)n1]

2-fluoro-4-(3-nitropyrazol-1-yl)pyridine (10.9 g; 48.04 mmol) was dissolved in a mixture of THF (100 mL) and methanol (50 mL) and was hydrogenated at 100 bar/‘full’ hydrogen at 70° C. at a flow rate of 0.7 mL/min using a ThalesNano CatCart® catalyst cartridge system, 70 mm (10% Pt/C) (ThalesNano Part Number THS-01133) in a ThalesNano H-Cube Pro™ flow reactor. The resulting product suspension was concentrated in vacuo to furnish 1-(2-fluoro-4-pyridyl)pyrazol-3-amine (8.11 g, 95%). ESI-MS m/z calc. 178.06548, found 178.9 (M+1)+.

### Step 3: Preparation of 1-(2-fluorophenyl)cyclopropanecarbonyl Chloride

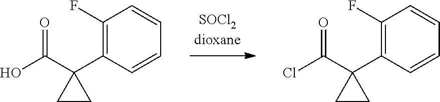 [SMILES: O=C(O)C1(c2ccccc2F)CC1 ; O=C(Cl)C1(c2ccccc2F)CC1]

In an Erlenmeyer flask, 27.71 mmol of 1-(2-fluorophenyl)cyclopropane-1-carboxylic acid was dissolved in 5 volumes of dioxane to afford a 1.11 M solution. In another flask, 72.15 mmol of thionyl chloride was dissolved in 2 volumes of dioxane to afford a 4.71 M solution. The solutions were combined at 2.6 equivalents of thionyl chloride at 150° C. in a Vapourtech® flow reactor with a 10 minute residence time. The product solution, containing 1-(2-fluorophenyl)cyclopropanecarbonyl chloride, was collected in a receiver flask.

### Step 4: Preparation of 1-(2-fluorophenyl)-N-(1-(2-fluoropyridin-4-yl)-1H-pyrazol-3-yl)cyclopropane-1-carboxamide

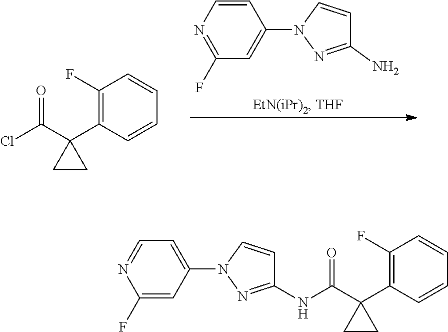

1-(2-fluoro-4-pyridyl)pyrazol-3-amine (5.10 g, 28.625 mmol, 1.000 equiv) was dissolved in anhydrous THF (152.6 mL, 31 vol), and ethylbis(propan-2-yl)amine (Hünig's base) (7.53 g, 10.2 mL, 58.280 mmol, 2.04 equiv) was added. Separately, 1-(2-fluorophenyl)cyclopropanecarbonyl chloride (6.18 g, 28.625 mmol, 1.000 equiv, 92.00% purity) was dissolved in anhydrous THF (58.4 ml, 11.4 vol). The two solutions were combined at 50° C. in a Vapourtech® flow reactor (2 mL reactor with a reagent line of 32 cm each) with a 5 minute residence time. The product solution, containing 1-(2-fluorophenyl)-N-(1-(2-fluoropyridin-4-yl)-1H-pyrazol-3-yl)cyclopropane-1-carboxamide, was captured in a reservoir.

While a number of embodiments of the invention have been described, the described embodiments may be altered to provide other embodiments within the scope of the invention. It will be appreciated that the scope of the invention is defined by the appended claims rather than by the specific embodiments that have been presented by way of example herein.

What is claimed is:

1. A process for preparing a compound of formula (I) 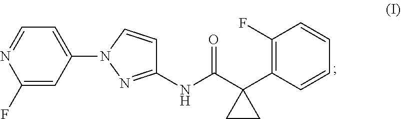 [SMILES: O=C(Nc1ccn(-c2ccnc(F)c2)n1)C1(c2ccccc2F)CC1]
or a pharmaceutically acceptable salt thereof, comprising:
reacting a compound of formula (II) 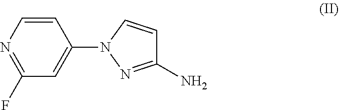 [SMILES: Nc1ccn(-c2ccnc(F)c2)n1]
with a compound of formula (III) 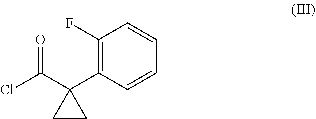 [SMILES: O=C(Cl)C1(c2ccccc2F)CC1]
in the presence of a suitable base to afford the compound of formula (I); and
optionally reacting the compound of formula (I) with a suitable acid to afford a pharmaceutically acceptable salt of the compound of formula (I).

2. The process of claim 1, wherein the base is a trialkylamine.

3. The process of claim 1 or 2, wherein the compound of formula (I) is purified by recrystallization from a mixture of isopropyl alcohol and water.

4. The process of claim 3, wherein the compound of formula (I) purified by recrystallization contains no more than 1 wt. % of the compound of formula (II).

5. The process of any one of claims 1 to 4, wherein the compound of formula (II) is obtained by reacting a compound of formula (IV) 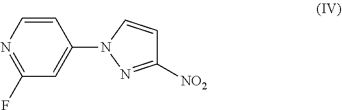 [SMILES: O=[N+]([O-])c1ccn(-c2ccnc(F)c2)n1]
with H₂ in the presence of a suitable hydrogenation catalyst to afford the compound of formula (II).

6. The process of claim 5, wherein the hydrogenation catalyst is a heterogeneous platinum catalyst.

7. The process of claim 5 or 6, wherein the compound of formula (II) is purified by recrystallization from a mixture of isopropyl alcohol and water.

8. The process of any one of claims 5 to 7, wherein the compound of formula (IV) is obtained by reacting a compound of formula (V) 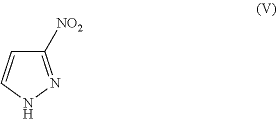
with a compound of formula (VI) 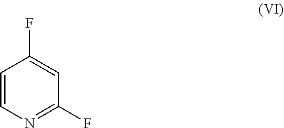
in the presence of a suitable base to afford the compound of formula (IV).

9. The process of claim 8, wherein the base is an inorganic phosphate or carbonate.

10. The process of claim 8 or 9, further comprising separating 2,4-bis(3-nitro-1H-pyrazol-1-yl)pyridine from the compound of formula (IV) by recrystallization from a mixture of dichloromethane and methanol.

11. A process for preparing a compound of formula (I) 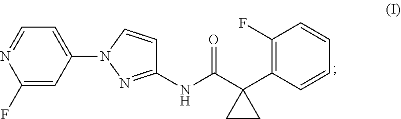 [SMILES: O=C(Nc1ccn(-c2ccnc(F)c2)n1)C1(c2ccccc2F)CC1]
or a pharmaceutically acceptable salt thereof, comprising:
reacting a compound of formula (V) 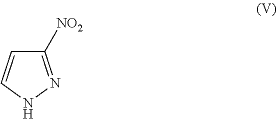
with a compound of formula (VI) 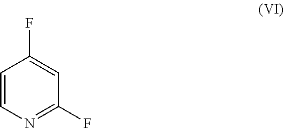
in the presence of a suitable base to afford a compound of formula (IV) 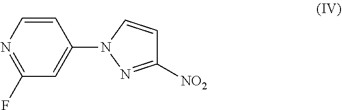 [SMILES: O=[N+]([O-])c1ccn(-c2ccnc(F)c2)n1]
and transforming the compound of formula (IV) to the compound of formula (I), or a pharmaceutically acceptable salt thereof.

12. The process of claim 11, wherein the base is an inorganic phosphate or carbonate.

13. The process of claim 11 or 12, further comprising separating 2,4-bis(3-nitro-1H-pyrazol-1-yl)pyridine from the compound of formula (IV) by recrystallization from a mixture of dichloromethane and methanol.

14. The process of any one of claims 11 to 13, wherein said transforming the compound of formula (IV) to the compound of formula (I), or a pharmaceutically acceptable salt thereof, comprises:
reacting the compound of formula (IV) with H₂ in the presence of a suitable hydrogenation catalyst to afford a compound of formula (II) 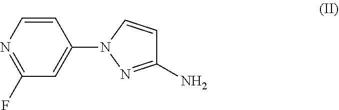 [SMILES: Nc1ccn(-c2ccnc(F)c2)n1]
and transforming the compound of formula (II) to the compound of formula (I), or a pharmaceutically acceptable salt thereof.

15. The process of claim 14, wherein the hydrogenation catalyst is a heterogeneous platinum catalyst.

16. The process of claim 14 or 15, wherein the compound of formula (II) is purified by recrystallization from a mixture of isopropyl alcohol and water.

17. The process of any one of claims 14 to 16, wherein said transforming the compound of formula (II) to the compound of formula (I), or a pharmaceutically acceptable salt thereof, comprises:
reacting the compound of formula (II) with a compound of formula (III) 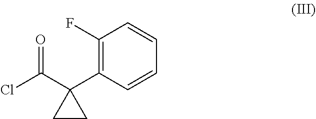 [SMILES: O=C(Cl)C1(c2ccccc2F)CC1]
in the presence of a suitable base to afford the compound of formula (I); and
optionally reacting the compound of formula (I) with a suitable acid to afford a pharmaceutically acceptable salt of the compound of formula (I).

18. The process of claim 17, wherein the base is a trialkylamine.

19. The process of claim 17 or 18, wherein the compound of formula (I) is purified by recrystallization from a mixture of isopropyl alcohol and water.

20. The process of claim 19, wherein the compound of formula (I) purified by recrystallization contains not more than 5 wt. %, 4 wt. %, 3 wt. %, 2 wt. %, 1 wt. %, 5000 ppm, 4000 ppm, 3000 ppm, 2000 ppm, 1000 ppm, 500 ppm, 250 ppm, 100 ppm, 50 ppm, 25 ppm, or 20 ppm of the compound of formula (II).

21. A process for preparing a compound of formula (I) 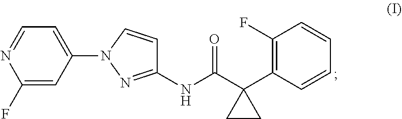 [SMILES: O=C(Nc1ccn(-c2ccnc(F)c2)n1)C1(c2ccccc2F)CC1]
comprising:
reacting a compound of formula (II) 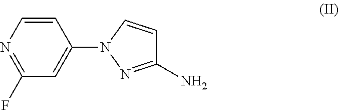 [SMILES: Nc1ccn(-c2ccnc(F)c2)n1]
with a compound of formula (III) 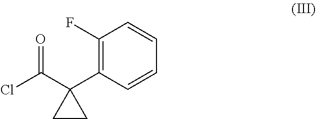 [SMILES: O=C(Cl)C1(c2ccccc2F)CC1]
in the presence of a suitable base to afford the compound of formula (I).

22. The process of claim 21, wherein the compound of formula (II) is obtained by reacting a compound of formula (IV) 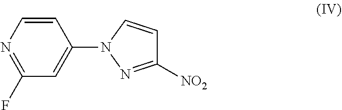 [SMILES: O=[N+]([O-])c1ccn(-c2ccnc(F)c2)n1]
with H₂ in the presence of a suitable hydrogenation catalyst to afford the compound of formula (II).

23. The process of claim 22, wherein the compound of formula (IV) is obtained by reacting a compound of formula (V) 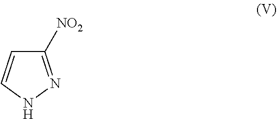
with a compound of formula (VI) 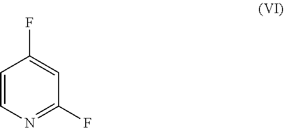
in the presence of a suitable base to afford the compound of formula (IV).

24. A process for preparing a compound of formula (I) 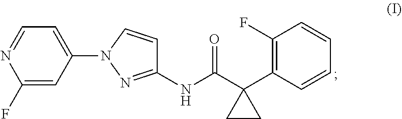 [SMILES: O=C(Nc1ccn(-c2ccnc(F)c2)n1)C1(c2ccccc2F)CC1]
comprising:
reacting a compound of formula (V) 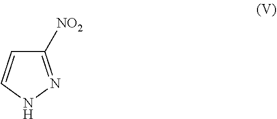
with a compound of formula (VI) 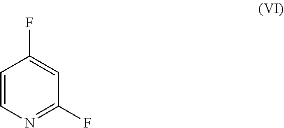
in the presence of a suitable base to afford a compound of formula (IV) 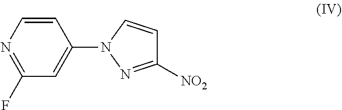 [SMILES: O=[N+]([O-])c1ccn(-c2ccnc(F)c2)n1]
and transforming the compound of formula (IV) to the compound of formula (I).

25. The process of claim 24, wherein said transforming the compound of formula (IV) to the compound of formula (I) comprises:
reacting the compound of formula (IV) with H₂ in the presence of a suitable hydrogenation catalyst to afford a compound of formula (II) 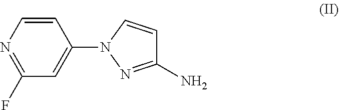 [SMILES: Nc1ccn(-c2ccnc(F)c2)n1]
and transforming the compound of formula (II) to the compound of formula (I).

26. The process of claim 25, wherein said transforming the compound of formula (II) to the compound of formula (I) comprises:
reacting the compound of formula (II) with a compound of formula (III) 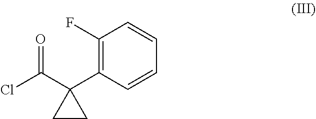 [SMILES: O=C(Cl)C1(c2ccccc2F)CC1]
in the presence of a suitable base to afford the compound of formula (I).

27. A compound of formula (II)

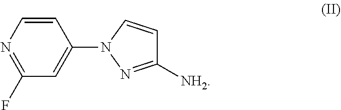 [SMILES: Nc1ccn(-c2ccnc(F)c2)n1]

28. A compound of formula (III)

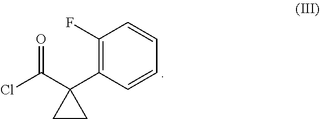 [SMILES: O=C(Cl)C1(c2ccccc2F)CC1]

29. A compound of formula (IV) 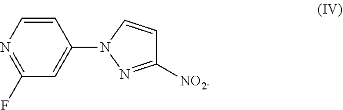 [SMILES: O=[N+]([O-])c1ccn(-c2ccnc(F)c2)n1]
